(19) United States
(12) Patent Application Publication
(10) Pub. No.: US 2023/0183776 A1
(43) Pub. Date: Jun. 15, 2023

(54) METHOD FOR REDUCING HOST NUCLEIC ACIDS IN BIOLOGICAL SAMPLE AND APPLICATIONS

(71) Applicants: GZ Vision Gene Technology Co., LTD., Guangzhou (CN); VISION MEDICAL DEVICES CO., LTD., Guangzhou (CN); VISION MEDICALS CLINICAL LABORATORIES CO., LTD., Guangzhou (CN); SZ VISION MEDICAL TECHNOLOGIES CO., LTD., Shenzhen City (CN); VISION MEDICALS CENTER FOR MEDICAL RESEARCHES CO., LTD., Shenzhen City (CN)

(72) Inventors: Teng XU, Guangzhou (CN); Weiqi ZENG, Guangzhou (CN); Junjie ZHANG, Guangzhou (CN); Yongjun LI, Guangzhou (CN); Xiaorui WANG, Guangzhou (CN); Hang SU, Guangzhou (CN)

(21) Appl. No.: 17/798,096

(22) PCT Filed: Nov. 23, 2020
(86) PCT No.: PCT/CN2020/130815

§ 371 Date: Jan. 31, 2023

(30) Foreign Application Priority Data
Feb. 12, 2020 (CN) 202010088891.6
Nov. 13, 2020 (CN) 202011271732.6

(51) Int. Cl. C12Q 1/6806; C12Q 1/04; C12Q 1/6869; C12Q 1/6888

(52) U.S. Cl. CPC C12Q 1/6806; C12Q 1/04; C12Q 1/6869; C12Q 1/6888

(57) ABSTRACT

A method of reducing host nucleic acids in a biological sample and a use thereof, which belong to the technical field of gene detection is provided. The method includes the steps of a) performing pre-treatment for the biological sample under a mild condition with partial lysis and/or no lysis of host cell membrane, thereby obtaining a liquid sample; and b) taking the liquid sample followed by adding a nucleic acid digestion reagent, thereby degrading the host nucleic acid which is exposed in the liquid sample. The method is used for reducing host nucleic acids in a biological sample and non-selectively enriching the targets, such as bacteria, fungi, viruses, mycoplasma/chlamydia.

### CROSS-REFERENCE TO RELATED APPLICATIONS

This application claims priority to PCT Application No. PCT/CN2020/130815, having a filing date of Nov. 23, 2020, which is based on Chinese Application No. 202010088891.6, having a filing date of Feb. 12, 2020, and Chinese Application No. 202011271732.6, having a filing date of Nov. 13, 2020, the entire contents all of which are hereby incorporated by reference.

### FIELD OF TECHNOLOGY

The following relates to the technical field of gene detection, particularly, it relates to a method of reducing host nucleic acids in biological sample and use thereof.

### BACKGROUND

Metagenomic sequencing (transcription) for pathogen is a new technology applied in clinical pathogen detection and has the advantages of being detectable for a wide range of pathogen, high sensitivity, high accuracy, and high effectiveness etc. Each clinical sample is generally sequenced with a sequencing data size of 20M in the present industry, and the rate of host nucleic acids to sample nucleic acids, averaged at 97%, affects the sensitivity of pathogen detection, hinders the cost reduction, and confines wider applications.

Metagenomic sequencing for pathogen always has a high rate of host nucleic acids to sample nucleic acids, mainly because of the following reason. The detection target is pathogenic microorganism in host, and the host (such as human) genome in the sample is 1000 times the size of the pathogenic microorganism (such as bacteria). In usual, the human genome size is about 3×10⁹ bp, while the bacterial genome size is about 3×10⁶ bp. Host (human) cells in the sample, even just a few of host cells, may lead to a rapid increase in the proportion of host nucleic acids. For the clinical samples taken from the infection part, the ratio of host to microbial is further increased, due to immune cell invasion.

Therefore, enriching the target detection object, i.e., pathogenic microorganism genome in samples, while removing host genome, has become the key factor to improve the detection efficiency of clinical pathogen infection.

For this purpose, various methods are used to increase the proportion of the pathogenic microorganism genome in the total genome of sample. For example, saponin is used to treat clinical samples to lyse human cells. However, saponin may cause instability of viral envelope containing cholesterol or mycoplasma membrane containing cholesterol, which affects the detection of some pathogens. Therefore, the use of saponin-treated samples in metagenomic sequencing for pathogen has obvious disadvantages. Alternatively, ionic surfactant or nonionic surfactant is used to lyse cells, because the ionic surfactant or nonionic surfactant, comprising sodium dodecyl sulfate (SDS), deoxycholic acid sodium salt, Triton X-100, NP-40 and the like, is often used as a lysis reagent for extracting bacterial nucleic acids. Alternatively, magnetic beads are complemented with the regions of repetitive sequences, i.e. Alu elements in the host genomic DNA, then Cas protein or its variants lacking the activity of endonuclease is induced to bind to the host genomic DNA, and then magnetic beads coated with streptavidin are added to capture the CRISPR-Cas system and its bound host DNA, thereby reducing the concentration of host genomic DNA in the sample solution. However, since the Alu regions accounts for about 10% of the total human genome sequence and Alu elements are not evenly distributed on the human genome, the effect of host removal is relatively limited.

### SUMMARY

An aspect relates to a method of reducing host nucleic acids in biological sample. This method can reduce the host nucleic acids in a biological sample and non-selectively enrich the targets, such as bacteria, fungi, viruses, mycoplasma, and chlamydia etc., which not only improves the detection sensitivity, but also reduces the detection cost.

A method of reducing host nucleic acids in a biological sample comprises steps of:

a) performing pre-treatment for the biological sample under a mild condition with partial lysis and/or no lysis of host cell membrane, thereby obtaining a liquid sample;

b) taking the liquid sample followed by adding a nucleic acid digestion reagent, thereby degrading the host nucleic acid which is exposed in the liquid sample.

At present, in gene detection, the Metagenomic sequencing method for pathogen has an influence on the sensitivity of pathogen detection, due to the high rate of host nucleic acids to sample nucleic acids. It was found that, in common technique, host cell membrane was lysed to expose the host genome and then the host nucleic acids were removed by degradation. For example, host nucleic acids were exposed, by using surfactants, such as saponin to differentially lyse host cell membrane, or by using pure water to lyse the cell membrane under osmotic pressure. However, in pathogenic microorganism detection, the above methods for lysing the host cell membranes may damage the structure of pathogenic microorganism, thereby affecting the detection of pathogenic microorganism and leading to a false negative result.

Further, when the pathogenic microorganism in clinical sample is detected, pathogen nucleic acids are mainly present in the form of pathogenic genome and pathogenic Cell-free DNA (cfDNA). The pathogenic Cell-free DNA (cfDNA) may be derived from the nucleic acids released by the pathogen lysis after the patient's immune system attacks the pathogen, or after antibiotics are used in clinic. Therefore, in the clinical molecular detection for pathogen, it is more significant to detect complete pathogen in clinic.

On the basis of the above research, it is proposed to expose host nucleic acids as much as possible under a mild pre-treatment condition, and then degrade the exposed host nucleic acids, without damaging pathogenic microorganisms. Thus, the above problem of high rate of false negative result is solved. At the same time, the integrity of pathogen can be ensured, enabling the detection result be significant for clinical reference.

According to the previous studies, it was found that, when all or most of host cell membrane could be lysed under a lysis condition, the lysis condition might inevitably affect the pathogenic microorganism. Therefore, the host cell membrane can only be partially lysed or not be lysed under a mild condition to ensure that the detection of pathogenic microorganism is not affected.

In one embodiment, the mild condition comprises the step of pre-treating the sample with a treatment solution having a relative concentration of 50% to 2200% in relative to isotonic solution. Alternatively, the mild condition comprises the step of performing ultrasonic treatment for the sample at a power of 0.1 W to 32 W, each treatment lasting for 0.1-10 min, with 0 to 30 s gap, 1 to 10 cycles. Alternatively, the mild condition comprises the step of striking the sample with a pressure of 0.1 to 1600 psi, each strike lasting for 0.1 to 15 min, with 0 to 30 s gap, 1 to 10 cycles. Alternatively, the mild condition comprises the step of treating the sample with 0.1 to 500 mg/L saponin or other detergents having a similar effect as saponin for 0.1 to 60 min.

It should be understood that, for the mild condition, when the sample is treated with a treatment solution having a relative concentration of 50% to 2200% in relative to isotonic solution, the relative concentration of 50% to 2200% defined means a working concentration of the sample solution in pre-treatment process, i.e., an environmental condition in which the host cells and microorganism are located, in the pre-treatment process. For example, if the volume of sample is small, the reagent concentration added to the sample can be set close to the working concentration; if the volume of sample is large, the reagent concentration added to the sample can be set to deviate from the working concentration, so as to improve the regulation ability after adding the sample.

The above 1 cycle means one treatment process. For example, when ultrasonic treatment is performed, 1 cycle means that the sample is treated by ultrasonic treatment at a power of 0.1 to 32 W for 0.1 to 10 min, thereby completing the treatment process.

Further, the mild condition comprises the steps of: performing ultrasonic treatment for the sample at a power of 13 W to 32 W, each treatment lasting for 2 to 7 s, with 5 to 15 s gap, 3 to 9 cycles. Alternatively, the mild condition comprises the steps of striking the sample with a pressure of 800 to 1200 psi, each strike lasting for 0.5 to 1.5 s, with 3 to 9 s gap, 5 to 15 cycles. Alternatively, the mild condition comprises the steps of treating the sample with 10 to 100 mg/L saponin or other detergents having the similar effect as saponin for 1 to 5 mins.

Further, the mild condition comprises the steps of performing ultrasonic treatment for the sample at a power of 0.1 W to 32 W, each treatment lasting for 4.5 s, with 9 s gap, 6 cycles. Alternatively, the mild condition comprises the steps of striking the sample with a pressure of 0.1 to 1600 psi, each strike lasting for is, with 6 s gap, 10 cycles. Alternatively, the mild condition comprises the steps of treating the sample with 0.1 to 100 mg/L saponin or other detergents having the similar effect as saponin for 1 to 2 mins.

It should be understood that the relative concentration refers to the concentration in relative to an isotonic solution, compared with plasma. For example, when 0.9% normal saline (i.e., isotonic solution) is regarded as 100% basal concentration, the treatment solution having the relative concentration of 50% to 2200% refers to 0.45% to 20% sodium chloride solution. The above isotonic solution refers to the solution having the same isotonic pressure as plasma, that is, 0.9% normal saline.

In previous studies, lysis test of leukocytes was performed by using normal saline in gradient concentrations, the data showed that when the relative concentration was 40% to 50%, complete lysis of leukocytes has reached to a critical point. That is, when the leukocytes were treated with sodium chloride solution having a concentration less than 0.45%, most of leukocytes were deactivated and lysed. Therefore, the pathogen cells could be less affected when the samples were treated with the above method.

In one embodiment, the mild condition comprises the steps of pre-treating the sample with a treatment solution having a relative concentration of 50% to 150% in relative to isotonic solution. In an embodiment, the treatment solution is at a relative concentration of 60% to 140%. In an embodiment, the treatment solution is at a relative concentration of 70% to 130%. In an embodiment, the treatment solution is at a relative concentration of 80% to 120%.

In one embodiment, in step a), samples are taken and pre-treated with a treatment solution at a relative concentration of 90% to 110% in relative to the isotonic solution. In an embodiment, the treatment solution is at a relative concentration of 95% to 105% in relative to the isotonic solution. In an embodiment, the treatment solution is at a relative concentration of 100% in relative to the isotonic solution. That is, the treatment is performed under a condition close to normal saline, so as to minimize the impact on pathogens.

It can be understood that, if the sample itself is a solution sample, it can be treated without adding any treatment solution, such as normal saline, etc. The sample is considered to be treated with 100% isotonic solution.

In one embodiment, the treatment solution is 0.45% to 20% sodium chloride solution or 2.5% to 7.5% glucose solution.

In one embodiment, the treatment solution is 0.45% to 1.35% sodium chloride solution.

In one embodiment, partially lysing or non-lysing the host cell membranes means that the proportion of living host cells is ≥0, detected by a Trypan blue staining method, an Annexin V cell apoptosis detection, or a method of staining living cells with PI. In an embodiment, the proportion of living host cells is >0. In an embodiment, the proportion of the living host cells is ≥10. In an embodiment, the proportion of the living host cells is ≥20%. In an embodiment, the proportion of living host cells is ≥50%.

It can be understood that, the proportion of the living cells mentioned above means the change of the proportion of the living cells after the mild treatment, compared with the proportion of the living cells before treatment. For example, if the proportion of living host cells is ≥20%, the proportion of living cells after mild treatment accounts for more than 20% of the total living cells before treatment.

In previous studies, it was found that, in order to ensure the integrity of pathogens, the evaluation of pathogen integrity was the most direct method. However, there was no good evaluation method for evaluating the pathogen integrity in the current technology. In practice, the critical conditions for complete cell lysis can be screened by evaluating the number of living host cells, so as to reduce the impact on pathogenic microorganism.

It can be understood that, the above methods for evaluating whether the host cell membrane is lysed are not exhaustive. Other evaluation methods, if available, may also be used, provided that the evaluation result, i.e., the integrity of host cell membrane, is the same as that obtained by the above method.

In one embodiment, the nucleic acid digestion reagents comprise nucleases and/or compounds that degrade nucleic acids.

In one embodiment, the nucleic acid digestion reagent is at least one or more selected from the group consisting of Benzonase, Turbo DNase, HL-SAN, DNase I or Propidium monoazide.

In one embodiment, in step 2), the nucleic acid digestion reagent is replaced with a nucleic acid blocker, so as to block free nucleic acids from entering subsequent detection steps.

The above nucleic acid blocker is used to affect/block the free/exposed nucleic acids from entering the subsequent process and then being detected, by degrading, coupling, and cross-linking, etc., and the nucleic acid blocker can be selected according to the experimental conditions.

In one embodiment, the biological sample is a biological sample that is suspected of infection in clinic.

In previous studies, it was found that, when biological samples was in a pathological state, the cells released nucleic acids into plasma to form cfDNA, due to 1) apoptosis, or 2) other pathological processes, such as necrocytosis and apoptosis induced by pathogen virulence proteins, or 3) necrocytosis mediated by pathogenic bacteria through inflammatory factors such as CIAS1/Cryopyrin/NLRP. On the other hand, when the organism was in an inflammatory state or other pathological states, neutrophil extracellular traps (NETs), i.e., activated neutrophil granulocytes could release decondensed chromatin to form a net scaffold, so as to surround and confine the pathogens. Such a process is called formation of neutrophil extracellular traps (NETosis). The NETs are a net consisted of DNA skeleton, histones, granular components and plasmosin. This net can capture and kill pathogens to participate in the body's immune response. The above factors all lead to an increase in the proportion of host extracellular nucleic acid. That is, in the pathological state, host nucleic acid has a natural “exposed” state. Compared with healthy biological samples, biological samples in pathological state naturally contain more host nucleic acids exposed in the solution. Alternatively, compared with cells in healthy biological samples, cells in a pathological state are more fragile and susceptible to damage by pre-treatment, because of the special state in the pathological process.

According to the above discoveries, it is suggested that, during the treatment of the biological samples in pathological state, even if the host cell membrane is not lysed, or is lysed under a very mild condition, the host nucleic acid in samples also can be well reduced, which greatly supports the present disclosure. That is, by pre-treatment under mild conditions, the host nucleic acids in biological sample can be reduced and the targets, such as bacteria, fungi, viruses, mycoplasma, and chlamydia, etc., can be non-selectively enriched, which not only improves the detection sensitivity, but also reduces the detection cost.

In one embodiment, the types of biological samples include: bronchoalveolar lavage fluid, cerebrospinal fluid, sputum, blood, pleural fluid, ascites, tissue, urine, pus, bone marrow, pericardial effusion, joint fluid, drainage fluid.

It is another aspect to provide a use of the method of reducing host nucleic acids in biological sample in a pathogenic microorganism detection.

The above method of reducing host nucleic acid in biological samples has very significant advantages, when applied in pathogenic microorganism detections. For example, it can be used in nucleic acid-based pathogen detection methods, including polymerase chain reaction (PCR), real-time fluorescence quantification PCR (qPCR), isothermal amplification (RPA, LAMP, etc.) and probe-based liquid phase capture detection methods or probe-based solid phase capture detection methods. Such application in these methods has the advantages of improving sensitivity and positive predictive value.

In one embodiment, the pathogenic microorganism detection is a microbial nucleic acid detection based on high-throughput sequencing, comprising the following steps:

a) treating a sample to be tested through the method of reducing host nucleic acids in biological sample mentioned above;

b) extracting nucleic acids from the sample;

c) constructing a library;

d) performing sequencing on a machine; and

e) analyzing data.

It is another aspect to provide a use of the method of reducing host nucleic acids in biological sample in a diagnosis of infectious diseases.

The above infectious diseases refer to a kind of disease caused by bacteria, fungi, viruses, mycoplasma/chlamydia and/or parasites.

It is another aspect to provide a kit for diagnosing infectious diseases, comprising:

a cell membrane lysis reagent, comprising a mild lysis reagent for partially lysing and/or non-lysing the cell membrane of host cells in biological sample;

a nucleic acid digestion reagent, comprising a nucleic acid remover for degrading free host nucleic acid or a nucleic acid blocker for blocking free nucleic acid from entering subsequent detection steps.

In one embodiment, the host cell membrane lysis reagent comprises a treatment solution at a relative concentration of 90% to 110% in relative to plasma isotonic solution, or 0.1 to 100 mg/L saponin or other equivalent reagents.

The nucleic acid digestion reagents comprise nucleases and/or compounds that degrade nucleic acids.

For example, the host cell membrane lysis reagent can be selected from 0.45% to 20% sodium chloride solution or other similar isotonic solution, and the nucleic acid digestion reagent is selected from Benzonase, Turbo DNase, HL-SAN, DNase I, or Propidium monoazide (PMA).

Compared with the conventional art, embodiments of the present disclosure has benefits including the following:

The method of reducing host nucleic acid in biological sample of embodiments of the present disclosure can expose the host nucleic acid as much as possible under a mild pretreatment condition, and then degrade the exposed nucleic acids, without damaging the pathogenic microorganism. Therefore, the method can not only solve the problem of high rate of false negative result in common technique, but also ensure the integrity of the pathogens at the same time, making the detection result be significant for clinical reference.

And it can be found in embodiments of the present disclosure that, when the biological sample is in a pathological state, the proportion of host extracellular nucleic acid was more than that of the biological sample in a healthy state. Alternatively, cells in a pathological state are more fragile than cells in healthy biological samples and susceptible to damage by pre-treatment, because of the special state in the pathological process. By utilizing the above cell characteristics cleverly, and by pretreatment under mild pretreatment conditions, the host nucleic acid in biological samples can be reduced and the targets, such as bacteria, fungi, viruses, mycoplasma/chlamydia, etc., can be enriched non-selectively, which not only improves the detection sensitivity, but also reduces the detection cost.

On the other hand, in general, the total quantity of the nucleic acids is small and therefore a few nucleic acids can be obtained, after host nucleic acids are removed by conventional methods, such as differential lysis. This has an impact on the subsequent construction of library and leads to low complexity of the library, and highly repetitive sequences, etc. However, compared with the common methods, the method of reducing host nucleic acid in biological sample of embodiments of the present disclosure have the advantages of less and simpler operation steps and short time-consumption, and the method can better solve the problem mentioned above, and can construct a library having high concentration and low repetitive sequences. Therefore, it is beneficial for stable detection result of metagenomics sequencing for pathogen in large-scale routine tests.

### BRIEF DESCRIPTION

Some of the examples will be described in detail, with reference to the following figures, wherein like designations denote like members, wherein:

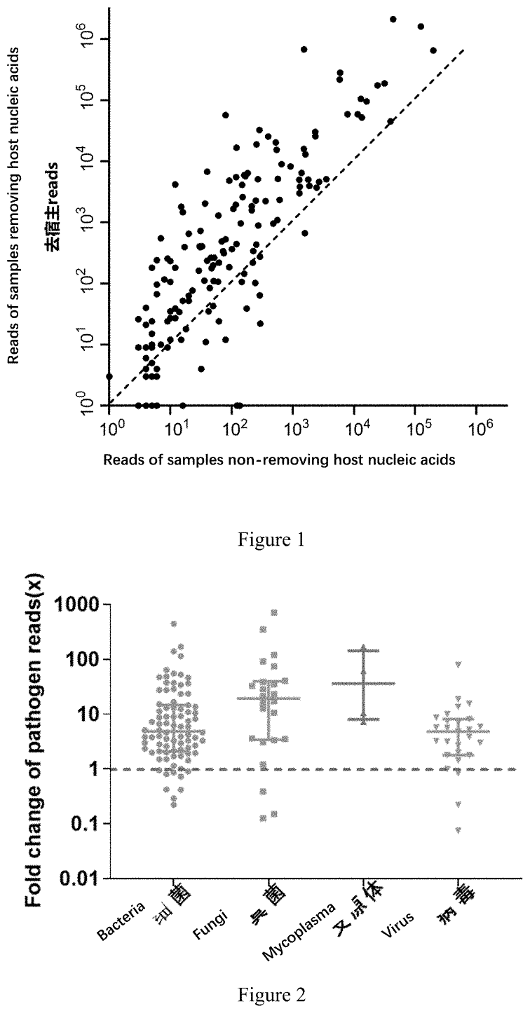

FIG. 1 shows a schematic diagram of analysis of reads difference between samples removing host nucleic acids and samples non-removing host nucleic acids, wherein the samples removing host nucleic acids are treated with normal saline;

FIG. 2 shows fold change of enriched pathogen reads for different kinds of pathogens;

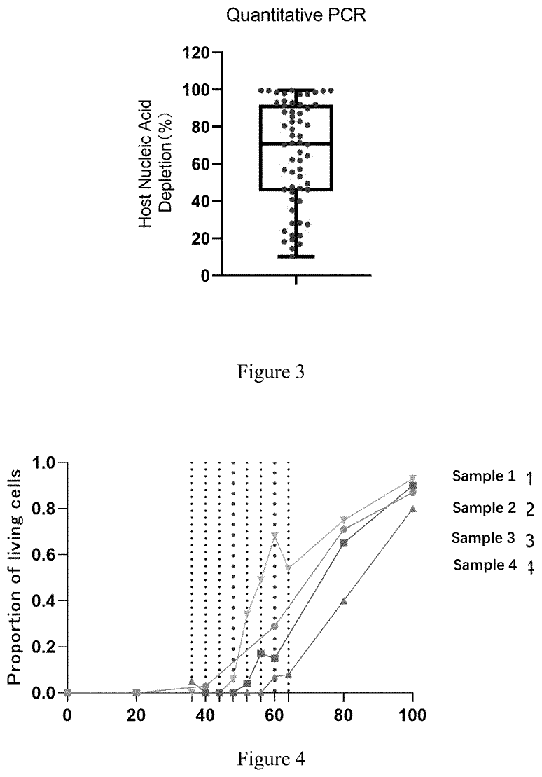

FIG. 3 shows a schematic diagram of evaluating the depletion proportion of host nucleic acid, by quantitative PCR (qPCR);

FIG. 4 shows a schematic diagram of effect of hypotonic normal saline in gradient concentrations on the lysis of leukocyte in Example 2;

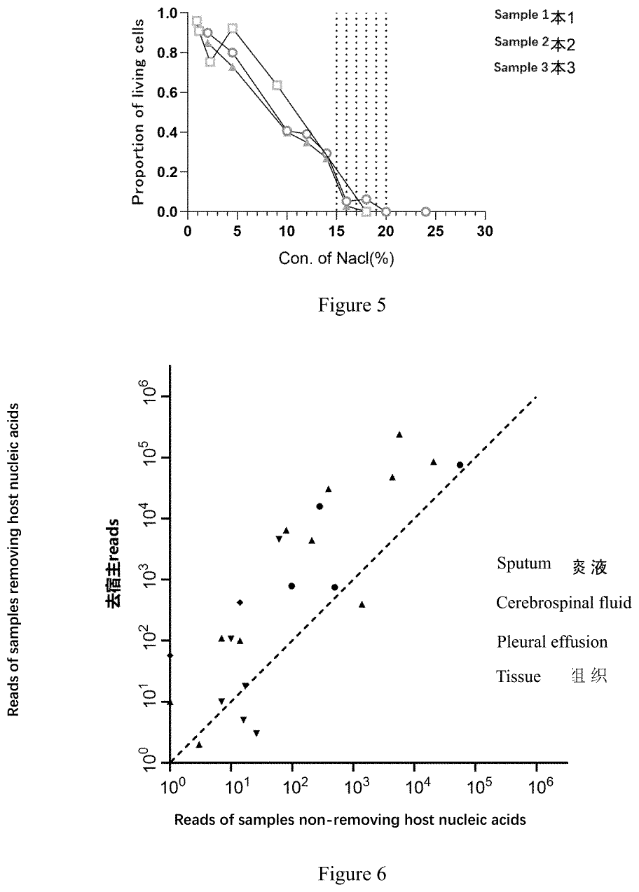

FIG. 5 shows a schematic diagram of effect of hypertonic normal saline in gradient concentrations on the lysis of leukocyte in Example 2;

FIG. 6 shows a schematic diagram of analysis of reads difference between samples removing host nucleic acids and samples non-removing host nucleic acids, for sputum, cerebrospinal fluid, pleural effusion, tissue samples; and

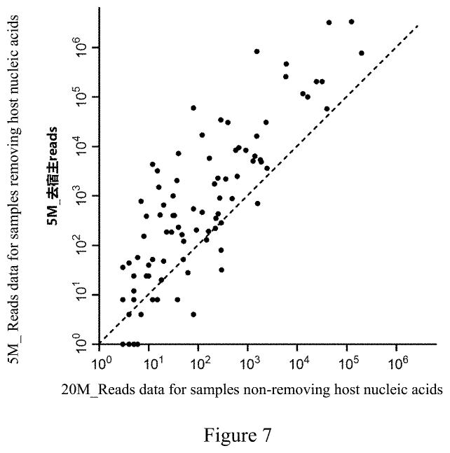

FIG. 7 shows a schematic diagram of analysis of reads difference between 5M data for samples removing host nucleic acids and 20 M data for samples non-removing host nucleic acids.

### DETAILED DESCRIPTION

For better understanding of the present disclosure, embodiments of the present disclosure will be fully described below in reference to relevant drawings. Some embodiments of the present invention are given in the drawings. However, the present disclosure can be implemented in many different forms and is not limited to the embodiments described herein. Rather, these embodiments are provided for the purpose of making the disclosed contents of the present disclosure more thorough and complete.

Unless otherwise defined, all technical and scientific terms used herein have the same meaning as those normally understood by one skilled in the conventional art in the technical field that the present disclosure is belonged to. The terms used in the description of the present disclosure herein are only for the purpose of describing specific embodiments, and are not intended to limit the present disclosure. The term “and/or” used herein refers to any one or more relevant items and their combination.

Unless otherwise specially mentioned, the raw materials used hereinafter are commercially available.

### Example 1

Samples were respectively treated with isotonic saline, by differential lysis, and by directly extracting nucleic acids. Results were compared.

(I). NGS Sequencing

I. Preparation of Samples

Fifty-seven bronchoalveolar lavage fluid (BALF) samples from patients (derived from patients suffering from lower respiratory infection) who were suspected of lower respiratory tract infection, were taken out from a freezer at 4° C., and three sub-samples were taken from each BALF sample and labelled, each sub-sample was 600 μl. The first sub-sample was treated with isotonic normal saline according to Method A (i.e., removing host nucleic acids by direct nuclease digestion) described as below and then nucleic acids were extracted. The second sub-sample was treated by a conventional differential lysis method according to Method B (i.e., removing host nucleic acids by differential lysis method) described as below and then nucleic acids were extracted. The third sub-sample was treated according to Method C (i.e., direct nucleic acid extraction) described as below, without removing host nucleic acids, and nucleic acids were directly extracted.

II. Pre-Treatment of the Samples

A. Direct Nuclease Digestion

1. The prepared samples were centrifuged at 12000 g for 5 min, and supernatant was removed, and 850 μl PBS was added for resuspending the samples. The PBS was isotonic 1×PBS (10 mmol/L Na₂HPO₄; 17.5 mmol/L KH₂PO₄; 137 mmol/L NaCl; 2.65 mmol/L KCl; pH 7.4)

2. 98 μl of 10× Benzonase Buffer and 2 μl of Benzonase (500 U) were added to the solution and the solution was incubated at 37° C. for 60 min.

3. After incubation, the solution was centrifuged at 12000 g for 3 min, and supernatant was removed and 50 μl liquid was remained.

4. 550 μl of 1×PBS was used for suspending the residues, and 6 μl of 0.5 M EDTA was added and mixed well.

B. Differential Lysis

1. The prepared samples were centrifuged at 12000 g for 5 min, supernatant was removed, and 850 μl of pure water was added for resuspending the samples, which were then incubated at room temperature for 10 min. The pure water was used for differentially lysing the samples, due to its hypoosmolality.

2. 98 μl of Benzonase Buffer and 2 μl of Benzonase (500 U) were added to the solution and the solution was incubated at 37° C. for 60 min.

3. After incubation, the solution was centrifuged at 12000 g for 3 min, and supernatant was removed.

4. 550 μl of 1×PBS was used for suspending the residues, and 6 μl of 0.5 M EDTA was added and mixed well.

C. Direct Nucleic Acid Extraction

The prepared samples were centrifuged at 12000 g for 5 min, supernatant was removed, 550 μl of 1×PBS was used for suspending the samples, and 6 μl of 0.5 M EDTA was added and mixed well.

III. Nucleic Acid Extraction

Nucleic acids were extracted for all samples according to the following process: DNA was extracted from BALF according to a method of extracting nucleic acid from micro-samples with genomic DNA extraction kit (DP316) manufactured by Beijing Tiangen Biochemical Technology Co., Ltd. The method of extracting nucleic acid comprises steps of:

1. taking 600 μl of the treated sample and adding it to a tube containing glass beads followed by lysing cell membrane with a physical vibrator.

2. after centrifugation for a short time, taking 300 μl of the treated samples and transferring it into a 1.5 ml tube; adding 10 μl of Proteinase K solution and then adding 100 μl of GB premixed with Carrier RNA (at a concentration of 1 μg/μl), and mixing the solution gently upside-down. After a centrifugation for a short time, removing liquid on the tube lid and tube inner walls;

3. keeping the sample in a warm bath at 56° C. for 10 min and gently shaking it occasionally;

4. adding anhydrous ethanol freeze-stored at −20° C. to the sample, followed by gently shaking the sample upside-down and placing the sample at room temperature for 3 min; after a centrifugation for a short time, removing liquid on the tube lid and tube inner walls;

5. adding the liquid obtained from the Step 4 into a CR₂ absorption column and centrifuging at 12000 rpm for 30 sec, removing waste streams, and then placing the CR₂ absorption column into a collection column.

6. adding 500 μl of Buffer GD solution to the CR₂ absorption column and centrifuging at 12000 rpm for 30 sec, removing waste streams, and then placing the CR₂ absorption column into a collection column;

7. adding 600 μl of Buffer PW solution to the CR₂ absorption column and centrifuging at 12000 rpm for 30 sec and removing waste streams, and then placing the CR₂ absorption column into a collection column.

8. repeating the Step 7;

9. centrifuging the solution at 12000 rpm for 30 sec and removing waste streams, and then placing the CR₂ absorption column into a collection column for 2 to 5 min at room temperature, to completely dry the residual rinse solution (Buffer PW solution) in the absorption material;

10. transferring the CR₂ absorption column into a clean centrifuge tube, dropwise adding 50 μl of elution Buffer TB to the middle of an adsorption film, placing the film at room temperature for 2 to 5 min and centrifuging it at 12000 rpm for 2 min to collect the solution into the centrifuge tube;

11. using Qubit 3.0 fluorometer (Thermo Fisher) to accurately quantify DNA sample concentration.

IV. Construction of Library

1. 5×TTBL and TTE Mix V1, stored at −20° C., were taken and thaw at 4° C. After complete mixing and centrifugation for a short time, magnetic beads, stored at 4° C., were taken out and placed at room temperature for 30 min until it reached equilibrium, and then the mixture was vibrated completely and mixed well, centrifuged for a short time for later use.

2. DNA fragmentation: a 20 μl reaction system was configured with 4 μl of 5×TTBL, 1 ng DNA, 5 μl of TTE Mix V1, supplemented with ddH₂O; the fragmentation reaction was performed in PCR according to the following procedures: heating lid at 105° C., reacting at 55° C. for 10 min, and storing at 10° C.

3. After reaction, 5 μl of 5×TS was added to the product obtained from the Step 2 and the mixture was gently blow with a pipettor and mixed well, placed at room temperature for 5 min.

4. PCR enrichment: a system described below was configured for the PCR enrichment. The system was configured with 25 μl of the product obtained from the Step 3, 10 μl of 5×TAB, 5 μl of upstream primers, 5 μl of downstream primers, and 1 μl of TAE, blow with a pipettor, and mixed well. The reaction tube was placed in a PCR machine, and the reaction was performed according to the following procedures: heating lid at 105° C., reacting a cycle (72° C. for 3 min, 98° C. for 30 sec, 98° C. for 15 sec, 60° C. for 30 sec, and 72° C. for 3 min), performing 5 to 15 cycles, and maintaining at 72° C. for 5 min, and storing at 4° C.

5. Sorting the product obtained from the Step 4: the magnetic beads equilibrated at room temperature in advance were used to sort the library fragments with an average length of 350 bp, according to the clean-up ratio of 0.7× beads used in first step and 0.15× beads used in the second step.

V. Sequencing on a Machine

The constructed library of nucleic acids was sequenced on Illumina NEXTSEQ550, with 20 M (20 million) reads per sample.

VI. Data Analysis

The sequenced data was analyzed and the result was shown hereinafter.

1. Enrichment Folds of Pathogen

Pathogen reads for fifty-seven bronchoalveolar lavage fluid (BALF) samples were obtained, and the pathogen reads for samples removing host nucleic acids were compared with those for samples non-removing host nucleic acids, which were shown in FIG. 1. Specifically, FIG. 1 showed analysis result of reads difference obtained by the metagenomic sequencing after treatment of BALF with Method A and Method C, respectively. From the figure, it could be seen that there is a correlation of pathogen detection for 57 BALF samples removing host nucleic acids (Method A) and non-removing host nucleic acids (Method C), wherein the abscissa is sequences reads for samples removing host nucleic acids, and the ordinate is sequences reads for samples non-removing host nucleic acids. The results showed that the pathogens of clinical samples were effectively enriched after removal of host nucleic acids.

FIG. 2 shows the analysis of the enrichment fold changes of different kinds of pathogens. It could be seen from the figure that, when adopting the above-mentioned direct nuclease digestion method (Method A) to treat samples, the average of enrichment folds of targets, such as bacteria, fungi, viruses and mycoplasma/chlamydia, etc., were 4.9 fold (Q1=2.12 fold, Q3=14.29 fold), 23.2 fold (Q1=3.46 fold, Q3=38.92 fold), 18 fold (Q1=9.64 fold, Q3=88.05 fold), 5.25 fold (Q1=1.95 fold, Q2=8.21 fold), respectively. The above Q1 refers to the quantile 25%, and Q3 refers to the quantile 75%.

2. Pathogen Loss Compared to the direct nucleic acid extraction method (Method C), when adopting the method of reducing host nucleic acid (Method A) in the example, no pathogen was lost, while enriching bacteria, fungi, viruses, mycoplasma/chlamydia, etc. Twenty-six bacteria, nine fungi, thirteen viruses and two chlamydia were detected in the 57 BALF samples.

However, when adopting the differential lysis method (Method B), Pseudomonas aeruginosa, Haemophilus parahaemolyticus, Stenotrophomonas maltophilia, etc. were lost in 4 samples. The results were shown in the following table.

TABLE 1
Sample reads with loss of Pseudomonas aeruginosa

| | Direct nucleic | | |
|---|---|---|---|
| | acid extraction | Differential | Reads |
| Sample M1942 | method (Reads) | lysis (Reads) | Ratio |
| Elizabethkingia | 12204 | 55569 | 4.55 |
| Stenotrophomonas | 4761 | 813 | 0.17 |
| mallophilia | | | |
| Pandora genus | 2795 | 636 | 0.23 |
| Acinetobacter baumanii | 338 | 244 | 0.72 |
| Pseudomonas aeruginosa | 146 | 0 | 0 |
| Human herpesvirus 5 | 66 | 41 | 0.62 |
| (CMV) | | | |
| Human herpesvirus 6B | 0 | 40 | 0 |
| Human herpesvirus 7 | 2 | 12 | 6 |

TABLE 2
Sample reads with loss of Haemophilus parahaemolyticus
and CMV

| | Direct nucleic | | |
|---|---|---|---|
| | acid extraction | Differential | Reads |
| Sample M1996 | method (Reads) | lysis (reads) | Ratio |
| Lawsonella_{—}clevelandensis | 647 | 301932 | 466.66 |
| Haemophilus parahaemolyticus | 13 | 0 | 0 |
| Hemophilus parainfluenzae | 6 | 912 | 152 |
| Human herpesvirus 5 (CMV) | 13 | 0 | 0 |
| Streptococcus parasanguis | 2 | 1363 | 681.5 |

TABLE 3
Sample reads with loss of Stenotrophomonas maltophilia

| | Direct nucleic | | |
|---|---|---|---|
| | acid extraction | Differential | Reads |
| Sample M1963 | method (reads) | lysis (reads) | Ratio |
| Enterococcus faecium | 434 | 130 | 0.3 |
| Stenotrophomonas | 28 | 0 | 0 |
| maltophilia | | | |
| Candida tropicalis | 41 | 76 | 1.85 |
| Aspergillus | 15 | 313 | 20.87 |
| Human herpesvirus 1 | 785 | 1423 | 1.81 |
| (CMV) | | | |
| Human herpesvirus 5 | 39 | 86 | 2.21 |
| (CMV) | | | |
| Human herpesvirus 4 | 1 | 7 | 7 |
| (EBV) | | | |

TABLE 4
Sample reads with loss of Pseudomonas aeruginosa

| | Direct nucleic | | |
|---|---|---|---|
| | acid extraction | Differential | Reads |
| M2003 | method (reads) | lysis (reads) | Ratio |
| Hemophilus | 374 | 13650 | 36.5 |
| parainfluenzae | | | |
| Pseudomonas | 355 | 0 | 0 |
| areuginosa | | | |

(II) qPCR Assay

### I. Method.

The fifty-seven BALF samples were treated according to the Method A (direct nuclease digestion method) and Method C (direct nucleic acid extraction) mentioned above and detected by qPCR assay using human specific primers (F: ATCAGCCACATTGGTCTCCTGGAG (SEQ ID NO: 1), R: GTGAGCCTTTGGGTTTGTCATTTGA (SEQ ID NO: 2)), each sample was taken in an equal amount of nucleic acids, with three replicates and the qPCR reaction system was performed according to the following table:

TABLE 5
qPCR Reaction System

| Reagent | Reaction system | |
|---|---|---|
| 2x μltraSYBR Mixture | 25 | μl |
| Forward Primer | 1 | μl |
| Reverse Primer | 1 | μl |
| Template DNA | 1.5-3 | ng |
| ddH₂O | Up to 50 | μl |

The qPCR reaction system configured as above was placed in an Applied biosystems 7500 system and the samples were amplified according to the parameters from the above table. The reaction was performed according to the following procedures: reacting a cycle (95° C. for 10 min, 95° C. for 15 sec, 60° C. for 1 min), performing such 40 cycles, and storing at 4° C.

### II. Results.

After the reaction, the differences of ΔCt between Method C (direct nucleic acid extraction) and Method A (direct nuclease digestion method), i.e., ΔCt results of nucleic acids of samples before and after removing host nucleic acids were shown in FIG. 3. FIG. 3 showed a schematic diagram of a depletion ratio of host nucleic acids evaluated by qPCR, wherein the ordinate was the proportion of host nucleic acid depletion. The proportion of host nucleic acid depletion was obtained by firstly calculating the ΔCt results of nucleic acids of samples before and after removing host nucleic acids (i.e., the nucleic acid depletion folds), and then converting ΔCt. The average of the proportion of host nucleic acid depletion reached 70%.

### Example 2

Treatment solutions at different isotonic relative concentrations have effects on sample treatment.

I. Leukocyte Lysis Assay with Gradient Normal Saline

1. Hypotonic Lysis Experiment.

The whole blood sample was taken and added into a centrifuge tube with an anticoagulant, centrifuged at 1600 g and separated to obtain a leukocyte layer. 0.9% sodium chloride saline was regarded to be at 100% relative concentration, and a series of gradient saline was prepared to lyse the leukocytes.

The results are shown in FIG. 4, wherein the abscissa is the relative concentration of the treatment solution, and the ordinate is the proportion of living cells counted by Trypan blue staining method. The results show that the lysis ratio of leukocyte decreases gradually, as the relative concentrations of the treatment solution increases. The lysis ratio of leukocyte is the lowest when the relative concentration of the treatment solution is 100%.

2. Hypertonic Lysis Experiment.

The whole blood sample was taken and added into a centrifuge tube with an anticoagulant, centrifuged at 1600 g and separated to obtain a leukocyte layer. Sodium chloride solution was prepared in gradient concentrations from 0.9 wt % to 24 wt % (being equivalent to 100% to 2660% relative concentration) to lyse the leukocytes.

The results are shown in FIG. 5, wherein the abscissa is the concentration of sodium chloride solution, and the ordinate is the proportion of living cells counted with Trypan blue as an indicator. The results show that, when the concentration (Con.) of sodium chloride solution reaches about 10 wt % (the relative concentration of sodium chloride solution is 1100%), the proportion of living cells decreases to 50 wt %; and when the concentration of sodium chloride solution reaches about 20 wt % (relative concentration is 2200%), the proportion of living cells decreases to 0, indicating that the cells can be completely lysed.

The above results show that, the lysis solution is 0.36%-20% of sodium chloride solution, or glucose or other salt solution having an equivalent concentration (similar lysis ability by osmotic pressure) as 0.36%-20% of sodium chloride solution. In an embodiment, the lysis solution is 0.54%-16% of sodium chloride solution, or glucose or other salt solution having an equivalent concentration as 0.54%-16% of sodium chloride solution. In an embodiment, the lysis solution is 0.63%-10% of sodium chloride solution, or glucose or other salt solution having an equivalent concentration as 0.63%-10% of sodium chloride solution.

II. Staining Assessment on the Leukocytes Lysed with Gradient Saline

A series of gradient normal saline was prepared respectively to lyse the leukocytes, according to the above method. Then the cell staining was performed and evaluated by a Trypan blue staining method, an Annexin V apoptosis detection method, and a method of staining living cells with PI, respectively. Specifically, the methods were shown as follows.

1. Trypan Blue Staining Method

Trypan blue is a dye used to distinguish living cells from dead cells. It is an important dye that is not absorbed by healthy living cells but stains cells with damaged cell membranes. It can be used to detect dead and dying cells, and cell viability. Specifically, the method comprises steps of.

1.1 taking 20 ul of Trypan blue and 20 ul of sample to be tested, respectively and mixing them in a ratio of 1:1.

1.2 incubating the cells at room temperature for 2 to 3 min.

1.3 detecting and counting the cells with optical microscope for microscopy.

2. Annexin V Apoptosis Detection Method

Annexin V is a reagent for detecting cell apoptosis. In normal cells, phosphatidylserine is only distributed on the inner side of the lipid bilayer of the cell membrane. During the early stage of cell apoptosis, the membrane phosphatidylserine (PS) is translocated from the inner side of the lipid membrane to the outer side of the lipid membrane. Annexin V as a phospholipid-binding protein has a high affinity for phosphatidylserine, and it binds to the cell membrane of early apoptosis cells through phosphatidylserine exposed on the outer side of the cell. Specifically, the method comprises steps of:

2.1 centrifuging 200 ul of the sample to be tested at 2000 rpm for 3 min, and removing a supernatant.

2.2 resuspending the sample with 400 ul of 1× Binding Buffer.

2.3 adding 5 ul of Annexin V-FITC to the cell suspension, mixing them gently and incubating at 2-8° C. for 15 minutes in the dark.

2.4 detecting the cells and counting with flow cytometer or fluorescence microscope.

3. Method of Staining Live Cells with PI

PI (Propidium iodide) is a nuclear staining reagent that can stain DNA and is often used for apoptosis detection. Although PI cannot pass through living cell membranes, it can pass through damaged cell membranes and stain nuclei. Specifically, the method comprises steps of:

3.1 washing 200 ul of sample to be tested twice with PBS.

3.2 resuspending the sample with 200 ul of 1×PBS.

3.3 adding PI dye (at a final concentration of 50 ug/ml) to the sample and incubating the sample at room temperature for 30 min in the dark.

3.4 detecting and counting the cells with flow cytometer or fluorescence microscope.

III. Treatment of Clinical Samples with Gradient Saline

1. Method.

Referring to the method of Example 1, five BALF samples were randomly selected, and three sub-samples were taken from each BALF sample and labeled, each sub-sample was 600 μl. The sub-sample pretreatment was carried out according to the methods in Example 1. For these three sub-samples, 850 μl of isotonic saline was added to the first sub-sample (Method D), and 850 μl of sodium chloride solution at a concentration of 0.54 wt % (i.e., normal saline at a relative concentration of 60%) was added to the second sub-sample (Method E), and 850 μl of sodium chloride solution at a concentration of 20 wt % (i.e., normal saline at a relative concentration of 2200%) was added to the third sub-sample (Method F).

2. Results

The results were shown as follows.

TABLE 6
Comparison of different treatment Methods

| | | Relative | Sequence | Relative | Sequence | Relative | Sequence |
|---|---|---|---|---|---|---|---|
| | | abundance of | number of | abundance of | number of | abundance of | number of |
| Sample | Pathogen | Method D | Method D | Method E | Method E | Method F | Method F |
| P1 | Acinetobacter | 17.32 | 458 | 19.48 | 708 | 20.32 | 540 |
| | baumanii | | | | | | |
| P1 | Stenotrophomonas | 19.64 | 341 | 4.92 | 115 | 13.68 | 326 |
| | maltophilia | | | | | | |
| P1 | Enterococcus | 0.97 | 28 | 0.84 | 35 | 0.72 | 18 |
| | faecium | | | | | | |
| P1 | Candida albicans | 89.45 | 264 | 90.27 | 113 | 92 | 120 |
| P2 | Streptococcus | 35.35 | 241 | 33.43 | 19468 | 45.61 | 13423 |
| | pneumoniae | | | | | | |
| P2 | Human herpesvirus-5 | 29.6 | 106 | 65.11 | 1297 | 44.3 | 404 |
| | (CMV) | | | | | | |
| P3 | Stenotrophomonas | 7.81 | 20 | 0.76 | 951 | 8.34 | 1006 |
| | maltophilia | | | | | | |
| P3 | Human herpesvirus-1 | 15.76 | 11 | 4.08 | 167 | 23.86 | 150 |
| | (HSV1) | | | | | | |
| P4 | Lactobacillus | 3.72 | 328 | 0.04 | 89 | 1.34 | 124 |
| | salivarius | | | | | | |
| P4 | Lactobacillus | 0.52 | 50 | 0.03 | 68 | 0.96 | 90 |
| | gasseri | | | | | | |
| P4 | Chlamydia | 1.6 | 65 | 0.08 | 82 | 2.45 | 230 |
| | psittaci | | | | | | |
| P5 | Pneumocystis | 0.7 | 31 | 42.76 | 63 | 30.08 | 70 |
| | jiroveci | | | | | | |

It can be seen from the above results that the enrichment folds for pathogenic microorganisms, as well as their abundance can be further increased by the direct nuclease digestion method in combination with hypotonic and mild lysis treatment. However, for some pathogenic microorganisms in the samples, their abundance decreases after the above direct nuclease digestion treatment in combination with the hypotonic and mild lysis treatment. This may be caused by drug administration or immune system attack, which makes some pathogens fragile, leading to a decrease in abundance. And it is further deduced that, using the differential lysis method may easily lead to pathogen loss, with the increasing lysis intensity.

### Example 3

The samples were treated with an ultrasonic method, a high-pressure method, and a chemical method, compared with those treated with sodium chloride solution at a relative concentration of 6000.

1. Method

Three BALF samples prepared in advance were taken (50 ul for each sample was reserved as a Trypan blue staining control), and eight sub-samples were taken from each BALF sample and labelled, each sub-sample was 600 μl. The first sub-sample was treated according to Method C of Example 1. The second sub-sample was treated according to Method E of Example 2. The third and fourth sub-samples were treated by using a sonicator (VCX130) (an ultrasonic method) with the following operating parameters: a 130 w power ultrasonic instrument, 10% or 25% power, 4.5 s sonication, with 9 s gap, 6 cycles. The fifth and sixth sub-samples were treated by using a high-pressure disrupter (Constant Systems) (high pressure method) with the following operating parameters: treated under 800 psi or 1200 psi for 5 s, with 6 s gap, 10 cycles. The seventh and eighth sub-samples were treated by adding 10 mg/L or 100 mg/L saponin (chemical method) and incubated at room temperature for 5 min.

After a mild lysis, the samples were treated and then nucleic acids were degraded according to the following steps:

1. adding 300 μl of 1×PBS to each sample treated by the ultrasonic method, the high-pressure method and the chemical method.

2. adding 98 μl of 10× Benzonase Buffer and 2 μl of Benzonase (500 U) to each treatment solution, 37° C., 60 min.

3. after incubation, centrifuging the samples at 12,000 g for 3 min, removing the supernatant and remaining 50 μl of sample liquid.

4. resuspending the sample with 550 μl of 1×PBS, adding 6 μl of 0.5M EDTA and mixing them well.

After the treated samples were obtained, the samples were divided into 2 parts, wherein 50 μl of the treated sample (one part) was stained with Trypan blue and the cells were counted and analyzed; for the remained 550p treated sample (the other part), sample nucleic acid extraction, library construction, computerization, etc. were carried out according to the method of Example 1.

II. Results Analysis

After treating the BALF by 60% mild hypotonicity method, the ultrasonic method, the high-pressure method and the chemical method, respectively, the samples were stained with Trypan blue according to the Example 2, and the cells were counted with an optical microscope. The results show that the proportion of living cells decreased with an increasing lysis intensity, as shown in Table 3.

TABLE 7
Cell counts under different lysis conditions

| | | | | Quantity | | |
|---|---|---|---|---|---|---|
| | | | | of living | Quantity | Propor- |
| | | | | cells in | of living | tion of |
| Treatment | | | Sample | a primary | cells after | living |
| methods | Condition | | ID | sample | treatment | cells |
| Mild | 60% | | P1 | 2.40E+05 | 4.50E+03 | 1.88% |
| hypotonicity | hypotonicity | | P2 | 7.86E+04 | 2.10E+03 | 2.67% |
| method | | | P3 | 6.48E+05 | 9.00E+03 | 1.39% |
| Ultrasonic | 10% | Power | P1 | 2.40E+05 | 5.46E+02 | 0.23% |
| method | | | P2 | 7.86E+04 | 1.07E+03 | 1.37% |
| | | | P3 | 6.48E+05 | 1.23E+04 | 1.90% |
| | 25% | Power | P1 | 2.40E+05 | 0.00E+00 | 0.00% |
| | | | P2 | 7.86E+04 | 3.43E+03 | 4.37% |
| | | | P3 | 6.48E+05 | 4.56E+03 | 0.70% |
| High- | 800 | psi | P1 | 2.40E+05 | 1.20E+04 | 5.00% |
| pressure | | | P2 | 7.86E+04 | 9.56E+03 | 12.17% |
| method | | | P3 | 6.48E+05 | 3.55E+04 | 5.48% |
| | 1200 | psi | P1 | 2.40E+05 | 2.30E+03 | 0.96% |
| | | | P2 | 7.86E+04 | 8.48E+03 | 10.79% |
| | | | P3 | 6.48E+05 | 4.37E+04 | 6.75% |
| Chemical | 10 | mg/L | P1 | 2.40E+05 | 1.10E+05 | 45.83% |
| method | | | P2 | 7.86E+04 | 4.68E+04 | 59.55% |
| | | | P3 | 6.48E+05 | 2.35E+05 | 36.24% |
| | 100 | mg/L | P1 | 2.40E+05 | 1.80E+03 | 0.75% |
| | | | P2 | 7.86E+04 | 4.65E+03 | 5.92% |
| | | | P3 | 6.48E+05 | 2.35E+04 | 3.62% |

The P1 sample of the above-mentioned treated sample was detected and analyzed with pathogen metagenomic detection according to Example 1, and the data analysis results are shown in the following table:

TABLE 8
comparison among different treatment methods

| | | | Hypotonicity | Ultrasonic | | High-pressure | | Chemical | |
|---|---|---|---|---|---|---|---|---|---|
| | | Non-removing | method | method | | method | | method | |
| | | host nucleic | 60% | 10% | 25% | 800 | 1200 | 10 | 100 |
| | | acids | hypotonicity | power | power | psi | psi | mg/L | mg/L |
| | Pathogen | Sequence | Sequence | Sequence | Sequence | Sequence | Sequence | Sequence | Sequence |
| #Sample | Name | number | number | number | number | number | number | number | number |
| P1 | Streptococcus | 20 | 75 | 83 | 98 | 57 | 63 | 12 | 78 |
| | oralis | | | | | | | | |
| P1 | Streptococcus | 13 | 68 | 64 | 107 | 125 | 120 | 35 | 76 |
| | pneumoniae | | | | | | | | |
| P1 | Staphylococcus | 0 | 17 | 23 | 16 | 34 | 23 | 5 | 60 |
| | aureus | | | | | | | | |
| P1 | Candida albicans | 145 | 304 | 245 | 305 | 236 | 290 | 412 | 450 |
| P2 | Klebsiella | 15 | 37 | 117 | 196 | 56 | 187 | 44 | 112 |
| | pneumoniae | | | | | | | | |
| P2 | Human herpesvirus-5 | 64 | 16 | 354 | 1559 | 453 | 506 | 83 | 1392 |
| | (CMV) | | | | | | | | |
| P3 | Acinetobacter | 4672 | 12011 | 26732 | 45655 | 35636 | 32468 | 15673 | 46738 |
| | baumanii | | | | | | | | |
| P3 | Enterococcus | 6 | 39 | 43 | 183 | 56 | 88 | 37 | 68 |
| | avium | | | | | | | | |
| P3 | Enterococcus | 0 | 6 | 5 | 27 | 23 | 18 | 13 | 34 |
| | faecium | | | | | | | | |
| P3 | Candida albicans | 7 | 22 | 85 | 60 | 56 | 67 | 5 | 24 |
| P3 | Candida glabrata | 8 | 18 | 45 | 82 | 23 | 107 | 21 | 67 |
| P3 | Human herpesvirus- | 0 | 13 | 76 | 34 | 16 | 22 | 7 | 39 |
| | 4(EBV) | | | | | | | | |

It can be seen from the above results that, compared with samples treated by the method of non-removing host nucleic acids (Method C in Example 1), some pathogens are obviously enriched in samples treated by the above methods, such as the mild lysis method with 60% hypotonicity, the ultrasonic method, the high-pressure method, and the chemical method, without any pathogen loss. However, there is no significant difference in pathogen sequence number for samples treated by the ultrasonic method, the high-pressure method and the chemical method, compared with those samples treated by 60% hypotonicity.

### Example 4

Different types of samples were treated with isotonic normal saline.

1. Methods.

Three samples of prepared sputum, cerebrospinal fluid, pleural effusion, and tissue were collected in advance, respectively, and two sub-samples were taken from each sample and labelled, each sub-sample was 600 μl. The first sub-sample was pretreated with the method A of Example 1 and then nucleic acids were extracted. The second sub-sample was treated with method C of Example 1, and nucleic acids were directly extracted without removing host nucleic acids.

Nucleic acid extraction and library construction were carried out according to Example 1, and the obtained library was sequenced using Illumina NEXTSEQ550; each sub-sample was sequenced with a data of 20M reads. The data were analyzed using the same Metagenomic sequencing method for pathogen as that in Example 1.

2. Results.

The results were shown in FIG. 6. The analysis diagram of the reads difference between samples removing host nucleic acids and samples non-removing host nucleic acids for the sputum (triangle), cerebrospinal fluid (circle), pleural effusion (diamond), and tissue (inverted triangle) samples. It can be seen from the figure that, the method of reducing host nucleic acid in biological samples in Example 1 is suitable for different types of biological samples, and they all have good enrichment of pathogenic microorganism nucleic acid. The read ratio of pathogen between samples removing host nucleic acids and samples non-removing host nucleic acids for these 12 samples was counted, and the median of enrichment fold was 8 times (Q1=1.35, Q3=21). In summary, the method of reducing host nucleic acids in biological samples is suitable for a variety of different samples, and has the effect of reducing host nucleic acid.

### Example 5

Pathological and healthy samples, both treated with isotonic saline, were compared.

1. Method

Three BALF samples in pathological state were taken, and Hela cells were used to simulate BALF samples at cell concentrations of 10⁶, 10⁵, and 10⁴, respectively. Two sub-samples were taken from each BALF sample and labelled, and each sub-sample was 600 μl. The first sub-sample was pre-treated according to the Method A of Example 1 followed by extracting nucleic acids. The second sub-sample was treated according to the Method C of Example 1 and followed by directly extracting nucleic acids, without removing the host nucleic acids, wherein the step of extracting nucleic acids referred to Example 1. The obtained nucleic acids were evaluated according to the qPCR method in Example 1, and the results for samples in pathological state and samples in simulated healthy state, treated by these two methods respectively, were compared.

2. Results.

The results are shown in the table below. After the BALF samples in the pathological state are treated with isotonic normal saline and digested with nuclease, the host nucleic acids decrease by 50%-90%, compared with samples non-removing host nucleic acids. However, for the BALF samples simulated by Hela cells, after a treatment with isotonic normal saline and then digestion with nuclease for removing the host nucleic acids, there is no significant difference in host nucleic acids, compared with samples without removing host nucleic acids. The results show that, when the clinical samples in pathological state and the simulated samples in healthy state are treated with isotonic normal saline, the clinical samples in pathological state have the effect of removing the host nucleic acids, but the samples in healthy state have no corresponding effect.

TABLE 9
Comparison of samples in pathological state
and simulated samples in healthy state

| | | | | Removing |
|---|---|---|---|---|
| | | Untreated | Treated | host nucleic |
| | | Samples | Samples | acids |
| | | average Ct | average Ct | (ΔCt) |
| BALF samples in | S1 | 24.87 | 26.96 | 2.09 |
| pathological state | S2 | 25.48 | 26.39 | 0.91 |
| | S3 | 28.89 | 32.9 | 4.01 |
| Samples in healthy state, | M1 | 21.45 | 21.61 | 0.16 |
| simulated by Hela cells | M2 | 24.83 | 24.7 | −0.13 |
| | M3 | 28.43 | 28.67 | 0.24 |

### Example 6

Currently, for metagenomic sequencing method for pathogen detection, most of the detection cost is in the sequencing. In the past, the sequencing industry assessed that the amount of sequencing data was required to reach 20M to achieve a balance between detection sensitivity and benefit.

In this example, the fastq files of 30 sub-samples from the 57 BALF samples in Example 1 were randomly intercepted and analyzed with 5M sequencing data, and compared with the detection results of the direct nucleic acid extraction method, i.e., Method C. The results are shown in FIG. 7, which is a schematic diagram of the analysis of reads difference between the 5M reads data for samples removing host nucleic acids and the 20M reads data for samples non-removing host nucleic acids. The results show that the pathogenic microorganisms are mainly distributed on the upper side of the diagonal, and there is no obvious loss of pathogenic microorganisms.

The above results show that, by removing host nucleic acids, the sequencing data of 5M is sufficient to achieve the sensitivity of conventional detection methods, and the sequencing cost is reduced to 25% of the conventional detection methods, which has significant economic benefits.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of reducing host nucleic acids in a biological sample, comprising:
a) performing a pre-treatment for the biological sample under a mild condition with at least one of partial lysis and no lysis of host cell membrane, thereby obtaining a liquid sample; and
b) taking the liquid sample followed by adding a nucleic acid digestion reagent, thereby degrading the host nucleic acid which is exposed in the liquid sample.

2. The method of reducing host nucleic acids in a biological sample of claim 1, wherein the mild condition comprises a step of pre-treating the sample with a treatment solution having a relative concentration of 50% to 2200% in relative to isotonic solution; or
wherein the mild condition comprises a step of performing ultrasonic treatment for the sample at a power of 0.1 W to 32 W, each treatment lasting for 0.1-10 min, with 0 to 30 s gap, 1 to 10 cycles; or;
wherein the mild condition comprises a step of striking the sample with a pressure of 0.1 to 1600 psi, each strike lasting for 0.1 to 15 min, with 0 to 30 s gap, 1 to 10 cycles; or
wherein the mild condition comprises a step of treating the sample with 0.1 to 500 mg/L saponin or other detergents having a similar effect as the saponin for 0.1 to 60 min.

3. The method of reducing host nucleic acids in a biological sample of claim 2, wherein the mild condition comprises steps of pre-treating the sample with a treatment solution having a relative concentration of 50% to 550% in relative to isotonic solution.

4. The method of reducing host nucleic acids in a biological sample of claim 3, wherein the mild condition comprises a step of pre-treating the sample with a treatment solution having a relative concentration of 50% to 150% in relative to isotonic solution.

5. The method of reducing host nucleic acids in a biological sample of claim 4, wherein in Step a), the biological sample is taken and pre-treated with a treatment solution having a relative concentration of 90% to 110% in relative to isotonic solution.

6. The method of reducing host nucleic acids in a biological sample of claim 5, wherein the treatment solution is 0.45% to 20% sodium chloride solution or 2.5% to 7.5% glucose solution.

7. The method of reducing host nucleic acids in biological sample of claim 6, wherein the treatment solution is 0.45% to 1.35% sodium chloride solution.

8. The method of reducing host nucleic acids in a biological sample of claim 1, wherein partial lysis or no lysis of the host cell membranes means that a proportion of host living cells is ≥0, detected by a Trypan blue staining method, an Annexin V cell apoptosis detection, or a method of staining living cells with PI.

9. The method of reducing host nucleic acids in a biological sample of claim 8, wherein partial lysis or no lysis of the host cell membranes means that the proportion of host living cells is >0, detected by a Trypan blue staining method, an Annexin V cell apoptosis detection, or a method of staining living cells with PI.

10. The method of reducing host nucleic acids in biological sample of claim 9, wherein partial lysis or no lysis of the host cell membranes means that the proportion of the host living cells is ≥20, detected by a Trypan blue staining method, an Annexin V cell apoptosis detection, or a method of staining living cells with PI.

11. The method of reducing host nucleic acids in a biological sample of claim 1, wherein the nucleic acid digestion reagents comprise at least one of nucleases and compounds that degrade nucleic acids.

12. The method of reducing host nucleic acids in a biological sample of claim 11, wherein the nucleic acid digestion reagent is at least one selected from the group consisting of Benzonase, Turbo DNase, HL-SAN, DNase I, or Propidium monoazide.

13. The method of reducing host nucleic acids in a biological sample of claim 11, wherein in step b), the nucleic acid digestion reagent is replaced with a nucleic acid blocker, blocking free nucleic acids from entering subsequent detection steps.

14. The method of reducing host nucleic acids in a biological sample of claim 1, wherein the biological sample is a biological sample that is suspected of infection in clinic.

15. The method of reducing host nucleic acids in a biological sample of claim 14, wherein the biological sample comprises bronchoalveolar lavage fluid, cerebrospinal fluid, sputum, blood, pleural fluid, ascites, tissue, urine, pus, bone marrow, pericardial effusion, joint fluid, drainage fluid.

16. A method of detecting pathogenic microorganism, comprising a step of reducing host nucleic acids in a biological sample.

17. The method of detecting pathogenic microorganism of claim 16, wherein the method of detecting pathogenic microorganism includes detecting microbial nucleic acid based on high-throughput sequencing, comprising:
a) treating a sample to be tested by performing a pre-treatment for the sample under a mild condition with at least one of partial lysis and no lysis of host cell membrane, thereby obtaining a liquid sample and taking the liquid sample followed by adding a nucleic acid digestion reagent, thereby degrading the host nucleic acid which is exposed in the liquid sample;
b) extracting nucleic acids from the sample;
c) constructing a library;
d) performing sequencing on a machine; and
e) analyzing data.

18. A method of diagnosing infectious diseases, comprising a step of reducing host nucleic acids in a biological sample.

19. A kit for diagnosing infectious diseases, comprising:
a cell membrane lysis reagent, comprising a mild lysis reagent for at least one of partially lysing and non-lysing the cell membrane of host cells in a biological sample; and
a nucleic acid digestion reagent, comprising a nucleic acid remover for degrading free host nucleic acid or a nucleic acid blocker for blocking free nucleic acid from entering subsequent detection steps.

20. The kit for diagnosing infectious diseases of claim 19, wherein the host cell membrane lysis reagent comprises a treatment solution at a relative concentration of 90% to 110% in relative to plasma isotonic solution, or 0.1 to 100 mg/L saponin or other equivalent reagents; and wherein the nucleic acid digestion reagents comprise nucleases and/or compounds that degrade nucleic acids.
